(19) United States
(12) Patent Application Publication
(10) Pub. No.: US 2012/0247187 A1
(43) Pub. Date: Oct. 4, 2012

(54) SENSOR MOUNTING INTO THE TEMPERATURE WELL OF A TRANSFORMER

(75) Inventor: Joshua J. Herz, Rochester, NY (US)

(73) Assignee: Qualitrol Company, LLC, Fairport, NY (US)

(21) Appl. No.: 13/077,082

(22) Filed: Mar. 31, 2011

(51) Int. Cl. G01N 33/00

(52) U.S. Cl. 73/31.06

(57) ABSTRACT

The invention provides a sensor assembly for a sensor having a semiconductor element for measuring hydrogen concentration in an insulating fluid in electric power generation, transmission, and distribution equipment having a temperature well that has a tubular portion extending into the equipment providing access to the interior of the equipment, the temperature well having a movable valve at an end of the tubular portion. The tubular portion includes a first flange, a tubular housing member attached to the first flange having one end adapted to be telescopically received in the temperature well. The tubular portion further includes a housing body having one end thereof connected to the tubular housing member having a substantially uniform cross section extending from the tubular housing member wherein at least one wire receiving opening extends through the housing body. The tubular portion also includes a cover closing an end of the housing body distal from the one end, a first seal disposed between the tubular housing member and the tubular portion of the temperature well for blocking the flow of insulating fluid in the space between the housing member and the temperature well wherein said tubular housing member is long enough so that when fully extended into the temperature well, the tubular housing causes the movable valve to open.

### CROSS-REFERENCE TO RELATED APPLICATIONS

None.

### FIELD OF THE INVENTION

This invention relates to the sensing of hydrogen in oils. It particularly relates to apparatus for sensing of hydrogen in electric power generation transmission and distribution equipment oil.

### BACKGROUND OF THE INVENTION

Electrical equipment, particularly medium-voltage or high-voltage electrical equipment, requires a high degree of electrical and thermal insulation between components thereof. Accordingly, it is well known to encapsulate components of electrical equipment, such as coils of a transformer, in a containment vessel and to fill the containment vessel with a fluid. The fluid facilitates dissipation of heat generated by the components and can be circulated through a heat exchanger to efficiently lower the operating temperature of the components. The fluid also serves as electrical insulation between components or to supplement other forms of insulation disposed around the components, such as cellulose paper or other insulating materials. Any fluid having the desired electrical and thermal properties can be used. Typically, electrical equipment is filled with an oil, such as castor oil, mineral oil, or vegetable oil, or a synthetic “oil”, such as chlorinated diphenyl or silicone.

Often, electrical equipment is used in a mission-critical environment in which failure can be very expensive, or even catastrophic, because of a loss of electric power to critical systems. In addition, failure of electrical equipment ordinarily results in a great deal of damage to the equipment itself and surrounding equipment thus requiring replacement of expensive equipment. Further, such failure can cause injury to personnel due to electric shock, fire, or explosion. Therefore, it is desirable to monitor the status of electrical equipment to predict potential failure of the equipment through detection of incipient faults and to take remedial action through repair, replacement, or adjustment of operating conditions of the equipment. However, the performance and behavior of fluid-filled electrical equipment inherently degrades over time. Faults and incipient faults should be distinguished from normal and acceptable degradation.

A known method of monitoring the status of fluid-filled electrical equipment is to monitor various parameters of the fluid. For example, the temperature of the fluid and the total combustible gas (TCG) in the fluid is known to be indicative of the operating state of fluid-filled electrical equipment. Therefore, monitoring these parameters of the fluid is used to maintain long life of the transformer. For example, it has been found that carbon monoxide and carbon dioxide increase in concentration with thermal aging and degradation of cellulosic insulation in electrical equipment. Hydrogen and various hydrocarbons (and derivatives thereof such as acetylene and ethylene) increase in concentration due to hot spots caused by circulating currents and dielectric breakdown such as corona and arcing. Concentrations of oxygen and nitrogen indicate the quality of the gas pressurizing system employed in large equipment, such as transformers. Accordingly, “dissolved gas analysis” (DGA) has become a well-accepted method of discerning incipient faults in fluid-filled electric equipment so as to maintain long life of the equipment.

In conventional DGA methods, an amount of fluid is removed from the containment vessel of the equipment through a drain valve. The removed fluid is then subjected to testing for dissolved gas in a lab or by equipment in the field. This method of testing is referred to herein as “offline” DGA. Since the gases are generated by various known faults, such as degradation of insulation material or other portions of electric components in the equipment, turn-to-turn shorts in coils, overloading, loose connections, or the like, various diagnostic theories have been developed for correlating the quantities of various gases in fluid with particular faults in electrical equipment in which the fluid is contained. However, since conventional methods of off-line DGA require removal of fluid from the electric equipment, these methods do not, 1) yield localized position information relating to any fault in the equipment, 2) account for spatial variations of gases in the equipment, and 3) provide real time data relating to faults. If analysis is conducted off site, results may not be obtained for several hours. Incipient faults may develop into failure of the equipment over such a period of time.

The measurement of hydrogen gas in the oil of an electrical transformer is of interest as it is an indication of the breakdown of the oil caused by overheating and/or arcing inside the transformer. Transformer oil cools the transformer and acts as a dielectric. As transformer oil ages it becomes a less effective dielectric. The increase in hydrogen dissolved in the transformer oil is an indicator of the coming failure of the transformer.

For large transformers there are hydrogen sensors that use gas chromatography or photo-acoustic spectroscopy to determine the amount of hydrogen gas within a transformer's oil. Such devices are very expensive and the expense is not justified for smaller transformers. There are many older, small transformers that could be monitored if a low-cost method of doing so was available.

A lower-cost gas monitor, the Hydran™ M2 manufactured by General Electric Company has been in use. However, this gas monitor only senses combustible gases and then uses a formula to estimate how much of the gas typically is hydrogen and how much is other gases.

An article “Overview of Online Oil Monitoring Technologies” by Tim Cargol at the Fourth Annual Weidmann-ACTI Technical Conference, San Antonio 2005 provides a discussion of oil gas measuring techniques, including hydrogen measurement.

Palladium hydrogen sensors are disclosed in Gases and Technology, July/August 2006, in the article, “Palladium Nanoparticle Hydrogen Sensor” pages 18-21. Palladium sensors are also disclosed in U.S. Patent Publications 2007/0125153—Visel et al., 2007/0068493—Pavlovsky, and 2004/0261500—Ng et al. U.S. Patent Application No. 2010/007828 discloses a hydrogen sensor for an electrical transformer.

There is a need for a low-cost method of determining hydrogen gas content in oils, such as in electric power generation and transmission and distribution equipment especially transformers. There is a particular need for a method and apparatus for mounting a hydrogen sensor to electric power generation transmission and distribution equipment that does not require taking the equipment out of service and preferably uses existing fittings or ports in the equipment without the necessity of making new openings in the housings for the equipment. It would particularly advantageous to provide a method and apparatus for attaching a hydrogen sensor to a transformer or the like using the port used for a pressure sensor especially a rapid pressure rise sensor.

### BRIEF SUMMARY OF THE INVENTION

The invention provides a sensor assembly for a sensor having a semiconductor element for measuring hydrogen concentration in an insulating fluid in electric power generation, transmission, and distribution equipment having a temperature well that has a tubular portion extending into the equipment providing access to the interior of the equipment, the temperature well having a movable valve at an end of the tubular portion. The tubular portion includes a first flange, a tubular housing member attached to the first flange having one end adapted to be telescopically received in the temperature well. The tubular portion further includes a housing body having one end thereof connected to the tubular housing member having a substantially uniform cross section extending from the tubular housing member wherein at least one wire receiving opening extends through the housing body. The tubular portion also includes a cover closing an end of the housing body distal from the one end, a first seal disposed between the tubular housing member and the tubular portion of the temperature well for blocking the flow of insulating fluid in the space between the housing member and the temperature well wherein said tubular housing member is long enough so that when fully extended into the temperature well, the tubular housing causes the movable valve to open.

### BRIEF DESCRIPTION OF THE SEVERAL VIEWS OF THE DRAWING(S)

FIG. 1 is a view of a transformer indicating possible locations for the attachments for sensors.

FIG. 2 is an expanded view of a sensor and its mount.

FIG. 3 is a cross sectional view of a sensor mounted on a transformer.

### DETAILED DESCRIPTION OF THE INVENTION

The invention provides numerous advantages over prior apparatus. The invention is smaller, easily installed, and lower in cost than other hydrogen sensing devices. The device is accurate and can be easily retrofitted onto existing transformers or engines. The device provides a very accurate hydrogen sensor with real time results as removal of fluid is not required. The device allows replacement of the sensor without providing a significant opening for oil to leave the container. The invention sensor utilizes instrument controls that are well known and available. These and other advantages will be apparent from the description below.

The invention provides easy retrofit of the hydrogen sensor to the transformer as an opening in the transformer housing is already present. This also is lower in cost than if a new inlet to the transformer needed to be installed. Further, the installation of the invention is low maintenance and will work at higher temperatures such as 120° C.

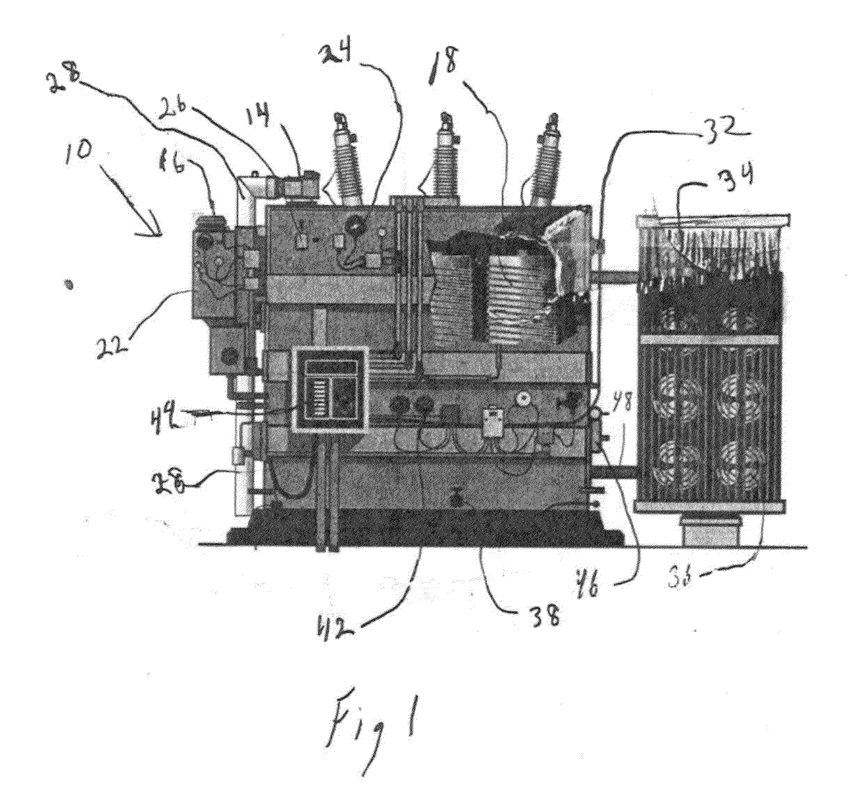

Illustrated in FIG. 1 is a transformer 10. The transformer 10 is provided with pressure relief devices 14 and 16. The transformer 10 is partially cutaway to show the coils 18. The transformer 10 has a temperature gauge 24. The temperature gauge 26 measures the temperature of the oil of the transformer. The pipe terminal 28 connects to the overflow pipe leading from pressure relief device 14. The optical fiber entry 32 provides direct reading of the winding temperature. A cooling tower 34 is utilized to regulate the temperature of the oil in the transformer by cooling when necessary using fans 36. The drain valve 38 is utilized to drain the oil for changing or to secure test samples. Electromechanical thermometers 42 sense the temperature of the oil in the transformer. The IED intelligent electronic device 44 controls the sensing devices and provides readouts of the information sensed. It further may control the cooling of the reactor as necessary. A rapid pressure rise relay 46 is also provided on the transformer. A flow gauge, not shown may be provided at location 48. The various temperature and pressure sensors, pressure release devices, drains, and flow gauges may provide mounting areas for hydrogen sensors.

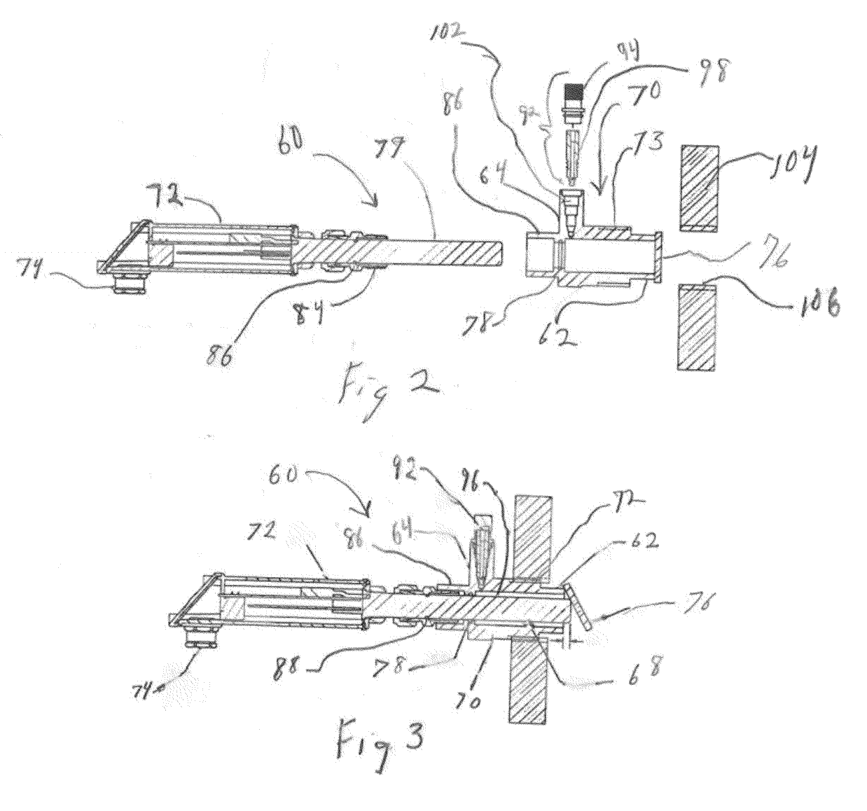

FIG. 2 and FIG. 3 illustrate the sensor assembly 60 of the invention having, in a preferred form, a semiconductor element for measuring hydrogen concentration in an insulating fluid in the electric power generation, transmission, and distribution equipment. The equipment 104 has a temperature well that has a tubular portion 62 extending into the electrical equipment 104 providing access through aperture 106 to the interior of the equipment. The temperature well has a movable valve 76 at the end of the tubular portion 62. The term temperature well is utilized as the preferred installation is in the location temperature sensors are located. However, the sensor apparatus 60 of the invention could be placed anywhere on a transformer where there is access through a tubular portion extending into the transformer fluid. The temperature well 70 of the sensor assembly comprises a first flange 64 and a tubular housing member 68 attached to the first flange having one end 77 adapted to be telescopically received in the temperature well 70. The housing body 72 has one end thereof connected to the tubular housing member 68 having a substantially uniform cross-section 73 extending from the housing body 72. There is an opening 74 for at least one wire to extend from the sensor assembly 60. There is a cover 76 over the distal end of the tubular portion 62. The cover 76 is located within the transformer or other electrical device. The cover 76 prevents leakage when the housing body 72 is removed from the transformer. A first seal 78 is disposed between the tubular housing member temperature well 70 and the tubular portion 62 of the temperature well 70 for blocking the flow of insulating fluid in the space between the housing member 72 and the temperature well 70 when the tubular portion 77 is inserted into the tubular portion 62. The tubular portion 77 of the housing member 72 is long enough that when fully extended into the temperature well the tubular housing causes movable valve 76 to open. The housing 77 has threaded portion 84 that allows it to be screwed into portion 106 of the temperature well 70. The threaded portion 84 housing 77 also contains at least one seal 88.

When the tubular housing member 77 is fully extended into the temperature well 70 the seal member 78 is positioned for engagement with both the tubular member 77 and the threaded portion 84. As illustrated in FIG. 3, the tubular member 77 when fully extended into the temperature well projects beyond the end of the tubular extension 62 and opens cover 76. The sensor assembly is designed such that when it is fully extended into the temperature well the tubular member projects to a point such that it does not interact with the electrically charged components of the equipment. Both the tubular portion 77 and the tubular housing 62 have a generally circular cross-section. The temperature well 70 is provided with a bleeder valve 92 that extends into the area 96 where fluid is present. The bleeder valve comprises a cap 94 and a body 98. It is threaded into the hole 102 and may be used to take fluid samples for measurement if desired.

The sensor assembly may be removed from the temperature well without significant leakage when the assembly is withdrawn. While the term temperature well has been utilized, it is intended that the tubular housing could be utilized for a variety of sensors. This should include sensors for temperature, pressure, hydrogen, carbon dioxide and other materials that may be in the cooling oil of electrical equipment. The apparatus as disclosed would allow use of the same aperture into a transformer for sensing a variety of things by changing the type of sensing apparatus. For instance, a hydrogen sensor could be utilized to sense the amount of hydrogen in the electrical equipment oil and then a pressure-measuring probe could be put in the same hole to measure the pressure in order to calculate the accurate hydrogen content. It is possible that the hydrogen probe also incorporate another sensor. In one preferred embodiment, the hydrogen probe also incorporates a temperature sensor so as to measure temperature and hydrogen with one probe.

The sensor assembly of the invention may be utilized in any portion of a transformer or other electrical device where there is an aperture through the wall. Such locations include the rapid pressure rise relay, the load tap changer, the drain valve, where electromechanical thermometers are present and where pressure relief valves are present. A preferred location is where the temperature sensors are located as they are installed in a tubular well such as illustrated herein.

Palladium containing hydrogen sensors and controllers for the sensors are known in the art. Such sensors are disclosed in United States Patent Publication Nos. 2007/0125153—Visel et al. and 2007/0240491—Pavlovsky, hereby incorporated by reference. An article in Gases and Technology, July/August 2006 “Palladium Nanoparticle Hydrogen Sensor” by I. Pavlovsky, also contains a description of hydrogen sensors and the methods and apparatus for their use. The palladium nanoparticles utilized in these preferred sensors for the invention are intrinsically sensitive to hydrogen and sensors based on palladium nanoparticle networks do not produce false alarms in the presence of other gases. This makes them particularly desirable for use in the devices of the invention as other gases may be present when the hydrogen is sensed. Other hydrogen sensors and their controllers are disclosed in U.S. Patent Publication Nos. 2007/0068493—Pavlovsky and 2007/0240491—Pavlosky et al., also incorporated herein by reference. The preferred hydrogen sensor for the instant invention is a semi-conductor palladium based hydrogen sensor because it gives accurate reading and can survive in the environment of the transformer.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. A sensor assembly for a sensor having a semiconductor element for measuring hydrogen concentration in an insulating fluid in electric power generation, transmission, and distribution equipment having a temperature well that has a tubular portion extending into the equipment providing access to the interior of the equipment, the temperature well having a movable valve at an end of the tubular portion thereof, comprising:
(a) a first flange;
(b) a tubular housing member attached to the first flange having one end adapted to be telescopically received in the temperature well;
(c) a housing body having one end thereof connected to the tubular housing member having a substantially uniform cross section extending from the tubular housing member;
at least one wire receiving opening extends through the housing body;
(d) a cover closing an end of the housing body distal from the one end;
(e) a first seal disposed between the tubular housing member and the tubular portion of the temperature well for blocking the flow of insulating fluid in the space between the housing member and the temperature well;
the tubular housing member being long enough so that when fully extended into the temperature well, the tubular housing causes the movable valve to open.

2. The sensor assembly of claim 1 wherein, with the tubular housing member fully extended into the temperature well, the seal member being positioned in engagement with both the tubular member and the tubular extension.

3. The sensor assembly of claim 1 wherein, with the tubular housing member fully extended into the temperature well, the tubular member projects beyond the end of the tubular extension.

4. The sensor assembly of claim 3 wherein, with the tubular housing member fully extended into the temperature well, the tubular member projects to a point such that it does not interact with electrically charged components of the equipment.

5. The sensor assembly of claim 3 in which the tubular portion and the tubular housing member each have a generally circular cross section.

6. The sensor assembly of claim 1 wherein the temperature well is provided with a sampling and bleeding valve.
